(19) United States
(12) Patent Application Publication
(10) Pub. No.: US 2023/0097015 A1
(43) Pub. Date: Mar. 30, 2023

(54) SELECTIVE SELF-ASSEMBLY BASED ARTIFICIAL METABOLON, PRODUCTION AND USE THEREOF

(71) Applicant: SEOUL NATIONAL UNIVERSITY R&DB FOUNDATION, Seoul (KR)

(72) Inventors: Yeo Joon YOON, Seoul (KR); Nam Ki Lee, Seoul (KR); Eunji Kim, Seoul (KR); Jin A Yoon, Seoul (KR); Soojung Yi, Seoul (KR); Sora Yang, Ansan-si (KR); Sun-Shin Cha, Seoul (KR); Ye-Eun Jung, Seoul (KR); Da-Woon Bae, Seoul (KR); Bo-Gyeong Jeong, Seoul (KR); Bu-Gyeong Kang, Seoul (KR); Ji-Sook Hahn, Seoul (KR); Sojeong Kim, Uiwang-si (KR); Injae Cho, Seoul (KR)

(73) Assignee: SEOUL NATIONAL UNIVERSITY R&DB FOUNDATION, Seoul (KR)

(21) Appl. No.: 17/937,111

(22) Filed: Sep. 30, 2022

(30) Foreign Application Priority Data
Sep. 30, 2021 (KR) 10-2021-0130290

(51) Int. Cl. C12P 13/04; C12N 15/62; C12N 15/52; C12N 15/81

(52) U.S. Cl. CPC C12P 13/04; C12N 15/62; C12N 15/52; C12N 15/81; C12Y 602/01012; C12Y 203/01074; C12Y 114/11022; C12N 2800/102

(57) ABSTRACT

The present disclosure relates to an artificial metabolon formed by selective self-assembly, production and use thereof, specifically, an artificial metabolon formed by de novo, in vivo assembly of multi-step metabolic pathway enzymes without using any external scaffold, production and use thereof.

### CROSS-REFERENCE TO RELATED APPLICATION

This application claims the benefit of Korean Patent Application No. 10-2021-0130290, filed on Sep. 30, 2021, in the Korean Intellectual Property Office, the disclosure of which is incorporated herein in its entirety by reference.

### INCORPORATION BY REFERENCE OF SEQUENCE LISTING

The content of the electronically submitted sequence listing, file name: Q280910_Sequence_Listing_as_ Filed.xml; size 17,985 bytes; and date of creating: Sep. 29, 2022, filed herewith, is incorporated herein by reference in its entirety.

### BACKGROUND

### 1. Field

The present disclosure relates to an artificial metabolon formed by selective self-assembly, production and use thereof, specifically, an artificial metabolon formed by de novo, in vivo assembly of multistep metabolic pathway enzymes without using any external scaffold, production and use thereof.

### 2. Description of the Related Art

Engineering metabolic pathways has been rapidly developed and used to produce various compounds, biofuels, pharmaceuticals, etc. from renewable resources. Much attention has been given to optimization of cellular metabolism, transcriptional control of enzyme expression, and optimization of enzyme activity through directed evolution in order to increase production yield of a target product, however, relatively little attention has been given to in vivo spatial organization of the enzymes constituting a metabolic pathway, or assembly of an artificial metabolon. The assembly of an artificial metabolon of biosynthetic enzymes related to a metabolic pathway allows substrate channeling and stoichiometric optimization of enzymes, and the substrate channeling has advantages of preventing accumulation of toxic metabolic intermediates, increasing the effective concentrations of intermediates, decreasing the transit time of intermediates, preventing loss of the intermediates by diffusion or competing metabolic pathways, protecting unstable intermediates from a solvent, and allowing bypassing a kinetically unfavorable equilibrium (Conrado R J et al. Curr Opin Biotechnol. 19(5):492-9 (2008)).

There has been an attempt to induce substrate channeling by direct fusion of proteins (Zhang Yet al. J Am Chem Soc. 128(40):13030-1 (2006)), however, fusing two or more enzymes may make it difficult to maintain the activity of the enzymes by causing non-functional folding of the proteins. On the other hand, methods of spatially localizing enzymes using various scaffolds have been developed, in which small peptides are attached to enzymes in metabolic pathways, and these peptides are recognized by scaffolds, which are structures composed of RNA, DNA, and proteins, so that the enzymes may exist in spatial proximity, and therefore, production yield of metabolic reactions may be improved. For example, it has been reported that production of resveratrol, 1,2-propanediol, and mevalonate increased via spatial arrangement of enzymes on a DNA scaffold by making a scaffold with DNA, and attaching a zinc finger peptide, which specifically recognizes and binds to a specific DNA sequence, to an enzyme (Conrado R J et al. Nucleic Acids Res. 40(4):1879-89 (2012)). However, DNA scaffolds are prone to plasmid recombination or supercoiling, making it difficult to control the spatial arrangement of enzymes, and RNA scaffolds are fragile and subject to misfolding, making it difficult to form a scaffold having multiple aptamers without structural disruption. Lastly, protein scaffolds based on specific interaction between signaling proteins and peptide ligands have been reported to enhance production of mevalonate, glucaric acid, etc (Dueber J E et al. Nat Biotechnol. 27(8):753-9 (2009)).However, expression of protein scaffolds may lead to metabolic burden on the production host cell due to the large size, and protein scaffolds are also prone to degradation and misfolding. In addition, there is also an issue of a limited number of signaling protein-peptide ligand pairs available in nature. Due to these issues, the scaffold systems developed so far have rarely been applied to enzymatic reactions of metabolic pathways comprised of more than three steps.

Accordingly, we have developed artificial metabolons formed by de novo, in vivo scaffold-free assembly of multistep metabolic pathway enzymes for universal applicability.

### SUMMARY

Provided herein is an artificial metabolon formed by selective self-assembly, comprising two or more enzyme modules.

Provided is a library of COM domain pairs that may be used to prepare the artificial metabolon formed by selective self-assembly.

Provided is a nucleic acid encoding the artificial metabolon formed by selective self-assembly, and a delivery system including the nucleic acid.

Provided is a cell including the nucleic acid encoding the artificial metabolon formed by selective self-assembly, and a method of preparing the artificial metabolon by using the cell.

Provided is a kit for preparation of the artificial metabolon formed by selective self-assembly.

Provided is a method of producing a target product by using the artificial metabolon formed by selective self-assembly.

### BRIEF DESCRIPTION OF THE DRAWINGS

FIG. 1 shows a schematic diagram of an artificial metabolon according to an embodiment of the present disclosure: COM^{D} and COM^{A} respectively refer to a donor COM domain and an acceptor COM domain present at the C-terminus and/or N-terminus of the enzyme (Enz).

FIG. 2 shows in vitro reaction results of a two-step artificial metabolon 4CL-CHS according to an embodiment of the present disclosure: productivity of naringenin is expressed as a peak area value in the UPLC-qTOF-HR-MS chromatogram.

FIG. 3 shows size exclusion chromatography (SEC) results showing interaction between 4CL-MlnB COM^{D} and MlnC COM^{A}-CHS constituting a two-step artificial metabolon according to an embodiment of the present disclosure: in the results of SEC, Peak1 corresponds to the complex formed by the interaction between 4CL-MlnB COM^{D} and MlnC COM^{A}-CHS, and 4CL-MlnB COM^{D} and MlnC COM^{A}-CHS bands in Peak1 were confirmed in 8% native polyacrylamide gel electrophoresis (PAGE) shown on the right.

FIG. 4 shows analysis of fluorescence resonance energy transfer (FRET) showing the interaction between 4CL-MlnB COM^{D} and MlnC COM^{A}-CHS constituting a two-step artificial metabolon according to an embodiment of the present disclosure.

FIG. 5 shows comparison of enzymatic activity of CHS to which a COM domain(s) according to an embodiment of the present disclosure is fused: activities according to the number of the COM domain(s) were compared as naringenin peak area values in the UPLC-qTOF-HR-MS chromatogram.

FIG. 6 shows comparison of enzymatic activity of FNS to which a COM domain(s) according to an embodiment of the present disclosure is fused: activities according to the type and number of the COM domain(s) were compared as conversion yield from naringenin to apigenin in an UPLC-qTOF-HR-MS analysis.

FIG. 7 shows a schematic diagram of three-step 4CL-CHS-FNS metabolon according to an embodiment of the present disclosure.

FIG. 8 shows combinations of recombinant enzymes for comparing effects of three-step 4CL-CHS-FNS metabolons according to an embodiment of the present disclosure: Metabolon control consists of free 4CL, CHS, and FNS that do not include any COM domain, Metabolon Assembly 1 consists of a metabolon comprised of 4CL and CHS, and free FNS, and Metabolon Assembly 2 consists of a metabolon comprised of 4CL, CHS, and FNS.

FIGS. 9A and 9B show in vitro reaction results of the three-step 4CL-CHS-FNS metabolon according to an embodiment of the present disclosure: FIG. 9A shows results of comparing productivity of naringenin, an intermediate in the three-step metabolic pathway, and FIG. 9B shows results of comparing productivity of apigenin, the final product in the three-step metabolic pathway.

FIG. 10 shows vectors for constructing a three-step in vivo artificial metabolon system according to an embodiment of the present disclosure: (a) is a schematic diagram of an operon including genes encoding enzymes constituting the three-step metabolon, with each enzyme as a free form (3-step_control), and (b) is a schematic diagram of an operon including genes encoding enzymes constituting the three-step metabolon, with each enzyme as a form fused to COM domains (3-step_assembly).

FIG. 11 shows vectors for constructing an in vivo artificial metabolon system in yeast according to an embodiment of the present disclosure: a two-step artificial metabolon was constructed by using two enzymes CNL and AAL, among the four enzymes DDGS-OMT-CNL-AAL in the pathway for biosynthesis of shinorine, a type of mycosporine-like amino acid (MAA): (a), (b), and (c) of FIG. 11 are schematic diagrams of genes encoding enzymes for the biosynthesis with each enzyme constituting the two-step metabolon as a free form (MAA metabolon control 1 to 3), and (d) is a schematic diagram of genes encoding enzymes for the biosynthesis with each enzyme constituting the two-step metabolon as a form fused to COM domains (MAA metabolon).

FIGS. 12A and 12B respectively show results of in vivo reaction of artificial metabolon at 48 hours and 120 hours of yeast culture according to an embodiment of the present disclosure: shinorine production in yeast having a metablon comprised of CNL and AAL, two enzymes in the shinorine biosynthesis pathway (MlnB COM^{D}-CNL-MlnC COM^{A}-AAL) was compared with that in yeast controls having CNL and AAL as a free form or a form fused to COM domain without forming a metabolon (CNL/AAL, MlnB COM^{D}-CNL/AAL, CNL/ MlnC COM^{A}-AAL) .

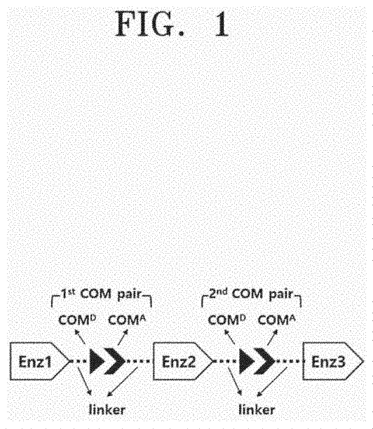

### DETAILED DESCRIPTION

Reference will now be made in detail to embodiments, embodiments of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below by referring to the figures, to explain aspects of the present description.

An aspect of the present disclosure provides an artificial metabolon formed by selective self-assembly, comprising 2 or more enzyme modules,

wherein the artificial metabolon is represented by (Xi)n,

Xi is an enzyme module consisting of N-terminal COM domain-recombinant enzyme-C-terminal COM domain,

i is an integer equal to or higher than 1, n is an integer equal to or higher than 2,

one of the N-terminal COM domain and the C-terminal COM domain is a donor COM domain (COM^{D}) and the other is an accepter COM domain (COM^{A}),

the donor COM domain (COM^{D}) of the (i)th enzyme module Xi specifically binds to an acceptor COM domain (COM^{A}) of the (i+1)th enzyme module Xi+1 so that all the enzymes from X1, a first enzyme module, to Xn, the (n)th enzyme module, are connected to form an artificial metabolon by selective self-assembly,

the first enzyme module X1 (i=1) of the artificial metabolon does not include any acceptor COM domain (COM^{A}), and the (n)th enzyme module (i=n) does not include any donor COM domain (COM^{D}), and

the reaction product produced by the recombinant enzyme of the (i)th enzyme module Xi is used as a substrate for the recombinant enzyme of the (i+1)th enzyme module Xi+1.

The term “artificial metabolon”, used herein, refers to an artificial enzyme chain or a multiple enzyme complex spatially reorganizing enzymes constituting an intracellular metabolic pathway in which multiple enzymatic reactions are continuously performed, for channeling and colocalization of substrate/intermediate metabolites. Artificial metabolon is defined as an enzyme cascade reaction system in which enzymes that continuously catalyze metabolic reactions are reconstituted to be in close proximity in a cell, by mimicking enzyme chains that occur in nature. Formation of an artificial enzyme chain increases productivity of cells by increasing flux through a biosynthesis pathway for producing metabolites, and final concentration of the desired product may be increased by reducing production of unwanted by-products. The term “artificial metabolon” is used herein interchangeably with “multi-enzyme cascade”, “artificial enzyme channel”, and the like.

We have developed a method of assembling multi-step enzymes without using a scaffold, by mimicking naturally-occurring large modular multifunctional enzyme complexes, for example, nonribosomal peptide synthetase (NRPS) and polyketide synthase (PKS). NRPS and PKS are composed of protein subunits, with each subunit comprising several modules, and each module responsible for a condensation reaction of an amino acid or acyl-CoA (Sussmuth R D and Mainz A. Angew Chem Int Ed Engl. 56(14):3770-3821 (2017); Kosol S et al. Nat Prod Rep. 35(10):1097-1109 (2018)). Modules of NRPS comprise a domain that selects and activates amino acids (adenylation domain), a domain that connects amino acids via a peptide bond (condensation domain), and a domain that tethers the peptide chain being synthesized (thiolation domain) (Dueber J E et al. Nat Biotechnol. 27(8):753-9 (2009)). Modules of PKS comprise a thiolation domain, a domain that selects a specific acyl-CoA to be incorporated into a polyketide chain in each module (acyltransferase domain), and a domain that forms a carbon-carbon bond (ketosynthase domain). PKS is divided into cis-AT PKS, in which the acyltransferase domain exists in the same protein as other domains, and trans-AT PKS, in which the acyltransferase domain exists independently (Kosol S et al. Nat Prod Rep. 35(10):1097-1109 (2018)). Sequence of condensation reactions in these modular multifunctional enzyme complexes depends on specific protein-protein interaction between protein subunits, and these interactions are mediated by a communication-mediating (COM) domain or a docking domain present at the termini of the subunits of NRPS or PKS. cis-AT PKS acts as a homodimer, but NRPS and trans-AT PKS are known to function as a monomer, and thus may be more suitably employed in assembling artificial metabolons. In addition, since the COM domain of NRPS consists of about 15 to 20 amino acids, and the docking domain of trans-AT PKS consists of about 25 amino acids, these domains are less likely to affect activities of enzymes to which they are fused.

The term “selective self-assembly”, used herein, refers to spontaneous assembly and spatial co-localization of enzymes via specific interactions between COM domains present at the N-terminus and/or C-terminus of each enzyme. Enzymes constituting a metabolon may be arranged in the order in which they act in a metabolic pathway, by the specific interactions between the COM domains.

The terms “COM domain” and “COM domain pair”, used herein, refer to domains and pairs thereof that are present at the N-terminus and/or C-terminus of an enzyme constituting a metabolon, and allow construction of a metabolon by selective self-assembly. A COM domain may be derived from the COM domains of NRPS or the docking domains of trans-AT PKS, and in the present specification, both the NRPS-derived COM domains and the trans-AT PKS-derived docking domain are referred to as a COM domain. Each COM domain recognizes and binds to a specific COM domain to form a pair of COM domains. COM domains at the N-terminus and/or C-terminus of each enzyme constituting a metabolon specifically interact to bind to respective relevant partner COM domains so that the enzymes are arranged from the N-terminus of the first enzyme to the C-terminus of the last enzyme in the metabolon. A COM domain present in an upstream enzyme may be referred to as a donor COM domain (COM^{D}), and a COM domain present in a downstream enzyme may be referred to as an accepter COM domain (COM^{A}) in a metabolon, and the enzymes are arranged to allow substrate/intermediate channeling and colocalization from upstream to downstream by interaction between the COM domains of the enzymes constituting the metabolon. Specifically, the first enzyme in a metabolon has a donor COM domain (COM^{D}) at either the N-terminus or the C-terminus, and no acceptor COM domain (COM^{A}); and the last enzyme has an acceptor COM domain (COM^{A}) at either the N-terminus or the C-terminus, and no donor COM domain (COM^{D}), and the enzymes between the first enzyme and the last enzyme have a donor COM domain (COM^{D}) at one of the N-terminus and C-terminus and an acceptor COM domain (COM^{A}) at the other terminus.

The term “enzyme module”, used herein, refers to a unit or a module including an enzyme that performs a step in the subject metabolic pathway implemented by a metabolon. Each enzyme module includes a recombinant enzyme having a COM domain at the N-terminus and/or C-terminus, and a linker may exist between the enzyme and the COM domain in the recombinant enzyme. The linker may be a peptide of a specific length, for example (GGGS)₂, and a length and composition of the linker may be selected to facilitate desired interactions between the enzyme and the COM domain, or to facilitate formation and/or activity of an artificial metabolon.

The term “recombinant enzyme”, used herein, refers to an enzyme produced by genetic recombination techniques to co-express a COM domain at the N-terminus and/or C-terminus of a naturally occurring enzyme, or the COM domain and a linker between the enzyme and the COM domain.

In an embodiment of the present disclosure, each enzyme module of the artificial metabolon may include a linker connecting the recombinant enzyme and the COM domain.

In an embodiment of the present disclosure, the linker connecting the recombinant enzyme and the COM domain may be GGGS, GGGGGS, or a multimer thereof, for example, (GGGS)₂, (GGGGS)₂, or (GGGGS)₃.

In an embodiment of the present disclosure, the donor COM domain (COM^{D}) of the enzyme module Xi and the acceptor COM domain (COM^{A}) of the enzyme module Xi+1 may have a predetermined binding affinity (Kd).

The term “binding affinity”, used herein, refers to a strength of the binding interaction between a single molecule, for example, protein or DNA, and a ligand or a binding partner thereof, and is measured as an equilibrium dissociation constant (Kd). The smaller the Kd value, the higher the binding affinity of the ligand to the target. Binding affinity is affected by non-covalent intermolecular interactions such as hydrogen bonding, electrostatic interactions, hydrophobic interactions, and Van der Waals forces between two molecules.

In an embodiment of the present disclosure, the pair of a donor COM domain (COM^{D}) and an acceptor COM domain (COM^{A}) that specifically bind to each other may be derived from the COM domains of NRPS or the docking domains of trans-AT PKS.

In an embodiment of the present disclosure, the pair of a donor COM domain (COM^{D}) and an acceptor COM domain (COM^{A}) may be selected from TycA-TycB, TycB-TycC, MlnB-MlnC, MlnD-MlnE, Kj12B-Kj12C, BaeM-BaeN, DifF-DifG, Ta-1-TaO, and RizD-RizE.

In an embodiment of the present disclosure, the artificial metabolon may include enzyme modules for the enzyme constituting a metabolic pathway for biosynthesis of a target product, and is capable of producing the target product from a starting substrate for the target product.

The term “target product”, used herein, refers to a final product to be produced using an artificial metabolon, and may be a final product of a naturally occurring biosynthetic metabolic pathway, or a final product of a metabolic pathway that has been modified or created by metabolic engineering using mutagenesis, genetic recombination, or gene editing. For example, the target product includes, but is not limited to, amino acids, peptides, proteins, lipids, monosaccharides, polysaccharides, nucleic acids, or compounds.

In an embodiment of the present disclosure, the artificial metabolon may include a plurality of identical enzyme modules. A plurality of the same enzyme module may be included, when needed to optimize biosynthesis of the target product and increase the yield.

Another aspect of the present disclosure provides an artificial metabolon,

which is an artificial metabolon for biosynthesis of apigenin, comprising:

a recombinant enzyme 4-coumarate CoA ligase (4CL) having a first COM domain at the N-terminus or C-terminus, a recombinant enzyme chalcone synthase (CHS) having a second COM domain and a third COM domain at the N-terminus and C-terminus, respectively, and a recombinant enzyme flavone synthase (FNS) having a fourth COM domain at the N-terminus,

wherein the first COM domain specifically binds to the second COM domain, and the third COM domain specifically binds to the fourth COM domain.

In an embodiment of the present disclosure, a linker may be included between the COM domain and the recombinant enzyme.

In an embodiment of the present disclosure, the pair of the first COM domain and the second COM domain and the pair of the third COM domain and the fourth COM domain may be a MlnB-C pair and a TycB-C pair, respectively.

In an embodiment of the present disclosure, the recombinant enzyme 4CL may consist of 4CL having an amino acid sequence of SEQ ID NO: 19, a (GGGGS)₂ linker located at the C-terminus of 4CL, and an MlnB COM^{D} domain linked thereto; the recombinant enzyme CHS may consist of CHS having an amino acid sequence of SEQ ID NO: 20, a (GGGGS)₃ linker located at the N-terminus of CHS, and an MlnB COM^{A} domain linked thereto, and a (GGGGS)₃ linker located at the C-terminus of CHS and a TycB COM^{D} domain linked thereto; and the recombinant enzyme FNS may consist of FNS having an amino acid sequence of SEQ ID NO: 21, a (GGGGS)₃ linker located at the N-terminus of FNS, and an TycC COM^{A} domain linked thereto.

Another aspect of the present disclosure provides an artificial metabolon

for biosynthesis of shinorine, comprising:

a recombinant enzyme ATP-grasp ligase (CNL) having a first COM domain at the N-terminus or C-terminus and a recombinant enzyme D-Alanine D-Alanine ligase (AAL) having a second COM domain at the N-terminus or C-terminus,

wherein the first COM domain specifically binds to the second COM domain.

In an embodiment of the present disclosure, a linker may be included between the COM domain and the recombinant enzyme.

In an embodiment of the present disclosure, the pair of the first COM domain and the second COM domain may be an MlnB-C pair.

Shinorine is a type of mycosporine-like amino acid (MAA) derived from microalgae, and is produced in a biosynthetic pathway consisting of four-step reactions by four enzymes, 2-desmethyl 4-deoxygadusol synthase (DDGS), O-methyltransferase (OMT), ATP-grasp ligase (CNL), and D-Alanine D-Alanine ligase (AAL). The yield of biosynthesis of shinorine was found to increase when a metabolon was formed with CNL and AAL by using COM domains, among the four enzymes involved in the biosynthesis.

In an embodiment of the present disclosure, the recombinant enzyme CNL may consist of CNL having an amino acid sequence of SEQ ID NO: 22, a (GGGS) linker located at the N-terminus of CNL, and an MlnB COM^{D} domain linked thereto, and the recombinant enzyme AAL may consist of AAL having an amino acid sequence of SEQ ID NO: 23, a (GGGGS)₂ linker located at the N-terminus of AAL, and an MlnC COM^{A} domain linked thereto.

A further aspect of the present disclosure provides a library of COM domain pairs which enables specific and selective interactions between enzymes to construct an artificial metabolon by selective self-assembly.

The library of COM domain pairs may be used to construct an artificial metabolon including enzymes involved in a multi-step metabolic pathway for producing a target product.

In consideration of the number of enzyme modules constituting the artificial metabolon, kinetics of each step, and an effect on the overall reaction, a COM domain pair having an appropriate length and binding affinity may be selected and used.

The library of COM domain pairs facilitates construction of artificial metabolons and makes them available for production of various target products.

In an embodiment of the present disclosure, COM domains constituting a COM domain pair may be modified as necessary. For example, COM domains may be modified by binding to a specific moiety or ligand in order to control binding affinity, to facilitate isolation of a target product, or to detect or monitor intermediates.

In an embodiment of the present disclosure, the COM domain pair library may include TycA-TycB, TycB-TycC, MlnB-MlnC, MlnD-MlnE, Kj12B-Kj12C, BaeM-BaeN, DifF-DifG, Ta-1-TaO, and RizD-RizE.

A further aspect of the present disclosure provides a recombinant vector comprising a nucleic acid encoding an artificial metabolon formed by selective self-assembly, wherein the recombinant vector includes an operon or an expression cassette in which genes respectively encoding each enzyme module are operably linked to expression regulatory sequences.

The artificial metabolon formed by selective self-assembly is as described above.

The term “operon”, used herein, refers to a genetic regulatory system consisting of a plurality of genes encoding enzymes constituting a metabolon and expression regulatory sequences thereof, in which expression of the enzymes is regulated by a single promoter.

The term “expression cassette”, used herein, refers to a nucleic acid construct including elements necessary for expressing a target product such as an enzyme constituting a metabolon, and includes an expression regulatory region including a promoter, and a gene encoding a target product and being operably linked to the expression regulatory region.

In an embodiment of the present disclosure, the recombinant vector may have a structure capable of controlling expression levels in consideration of stoichiometry of the enzymes constituting the artificial metabolon.

In an embodiment of the present disclosure, the recombinant vector is configured to have genes encoding enzyme modules constituting a metabolon between a promoter and a terminator, and may be reconstructed by selecting the number or order of genes, expression regulatory factors, and the like, in consideration of the number of the enzyme modules constituting a metabolon or stoichiometric contribution of a reaction by each enzyme module in a metabolic pathway.

In an embodiment of the present disclosure, the recombinant vector may be a pET vector, pGEX vector, pMAL vector, pTrc vector, pACYC vector, pCDF vector, or pRSF vector used for protein expression in E. coli.

In an embodiment of the present disclosure, the recombinant vector used for protein expression in E. coli may include a T7 promoter, a Tac promoter, or a Trc promoter.

In an embodiment of the present disclosure, the recombinant vector may be a CEN/ARS vector used for protein expression in yeast.

In an embodiment of the present disclosure, the recombinant vector used for protein expression in yeast may include a TDH3 promoter.

In an embodiment of the present disclosure, the recombinant vector may be configured to express with high efficiency in transformed cells to construct an artificial metabolon.

In an embodiment of the present disclosure, the recombinant vector includes a nucleic acid encoding a part of an artificial metabolon of interest, and may be used in conjunction with other recombinant vectors including nucleic acids encoding the remainder of the artificial metabolon.

In an embodiment of the present disclosure, nucleic acids encoding the artificial metabolon of interest may be included in a plurality of recombinant vectors.

Still another aspect of the present disclosure provides a cell transformed with a recombinant vector including a nucleic acid encoding the artificial metabolon of interest.

The term “transformation”, used herein, refers to introduction of a gene into a cell to express a foreign gene, and a cell may be transformed by introduction of a recombinant vector.

In an embodiment of the present disclosure, the cell may be a cell capable of expressing a metabolon, and may include, but is not limited to, E. coli, yeast, actinomycetes, or strains of Corynebacterium genus.

In an embodiment of the present disclosure, the transformed cell may be a cell capable of producing a target product by an artificial metabolon, without affecting self-assembly of the artificial metabolon with the nucleic acid encoding the artificial metabolon expressed.

In an embodiment of the present disclosure, the transformed cell may be a cell modified to efficiently express a nucleic acid encoding an artificial metabolon, facilitate self-assembly of the artificial metabolon, and produce a target product in high yield, for example, a cell with a high concentration of intracellular ATP or a precursor of the target product.

In an embodiment of the present disclosure, the transformed cell may be used for in vitro or in vivo production of a target product using the artificial metabolon.

Still another aspect of the present disclosure provides a method of producing a target product by using an artificial metabolon according to the present disclosure, including: contacting a substrate of the recombinant enzyme in the first enzyme module of an artificial metabolon with the artificial metabolon.

In an embodiment of the present disclosure, the method may be performed in vitro. Specifically, the method may be performed in vitro by culturing cells transformed with a vector including a nucleic acid encoding an artificial metabolon to express the enzymes constituting the metabolon, separating and purifying the enzymes by affinity chromatography, mixing the enzymes in order that artificial metabolons are formed in vitro by selective self-assembly, and contacting the artificial metabolons with a first substrate. After reactions by the artificial metabolons in vitro, the target product may be separated and recovered by extraction or chromatography.

In an embodiment of the present disclosure, the method may be performed in vivo, and may include culturing cells transformed with the nucleic acid encoding the artificial metabolon in the presence of the substrate. Specifically, the cells may be cultured in the presence of the substrate, so that a target product may be produced from the substrate by the artificial metabolon in the cell, and the target product may be isolated from the cell culture. After culturing the cells expressing the artificial metabolon, the target product may be separated and recovered by extraction, chromatography, or the like.

Culturing of the transformed cells may be performed under conditions that may maximize production of the artificial metabolon, and culture conditions and culture period may be determined as necessary by those skilled in the art to which the present disclosure pertains.

In an embodiment of the present disclosure, the target product includes, but is not limited to, amino acids, peptides, proteins, lipids, monosaccharides, polysaccharides, nucleic acids, or compounds.

According to the present disclosure, an artificial metabolon that implements a multi-step metabolic pathway may be assembled by using specific interactions between the COM domains of NRPS or the docking domains of trans-AT PKS, without an external scaffold, and useful compounds, proteins, biofuels, pharmaceuticals, etc. may be produced with high productivity via substrate/intermediate channeling.

### EXAMPLES

One or more embodiments of the present disclosure will be described in detail with reference to the following examples. However, these examples are for illustrative purposes only and are not intended to limit the scope of the one or more embodiments of the present disclosure.

### Example 1

### Preparation of Artificial Metabolon

1-1. Selection of COM Domain Pairs

9 types of communication-mediating (COM) domains or docking domains were selected from nonribosomal peptide synthetase (NRPS) and polyketide synthase (PKS), which are large modular multifunctional enzymes that exist in nature, in order to use the domains in the preparation of artificial metabolons. Among the 9 selected COM domain pairs were two pairs of COM domains derived from tyrocidin A synthetase of Bacillus brevis, a general NRPS with a typical biosynthetic mechanism, a TycA COM donor (COMD)-TycB COM acceptor (COMA) pair (TycA-B), and a TycB COMD-TycC COMA pair (TycB-C) (Hahn M and Stachelhaus T. Proc Natl Acad Sci USA. 101(44):15585-90 (2004)). One of the 9 selected COM domain pairs was a pair of COM domains derived from rhabdopeptide/xenortide-like peptide (RXP)-type NRPS of Xenorhabdus stockiae which acts atypically, a Kj12B COMD-Kj12C COMA pair (Kj12B-C) (Hacker C et al. Nat Commun. 9(1):4366 (2018)); and six were two pairs of COM domains derived from a macrolactin synthase, which is trans-AT PKS of Bacillus amyloliquefaciens, in which an AT domain that selects and delivers substrates exists and functions in a free state unlike cis-AT PKS, MlnB COMD-MlnC COMA pair (MlnB-C), and MlnD COMD-MlnE COMA pair (MlnD-E); a pair of COM domains derived from a bacillaene synthase, BaeM COMD-BaeN COMA pair (BaeM-N); a pair of COM domains derived from a difficidin synthase, DifF COMD-DifG COMA pair (DifF-G) (Zeng J et al. ACS Chem Biol. 11(9):2466-74 (2016)); a pair of COM domains derived from a myxovirescin synthase of Myxococcus xanthus, Ta-1 COMD-TaO COMA pair (Ta-1-O) (Simunovic V et al. Chembiochem. 7(8):1206-20 (2006)); and a pair of COM domains derived from a rhizopodin synthase of Stigmatella aurantiaca, a RizD COMD-RizE COMA pair (RizD-E) (Pistorius D and Muller R. Chembiochem. 13(3):416-26 (2012)).

The selected COM pairs are known to interact in a form of a monomer. The selected COM domains and the amino acid sequences thereof are listed in Table 1.

TABLE 1

| COM | | Number of | |
|---|---|---|---|
| pair | COM domain | amino acids | Amino acid sequence |
| TycA-B | TycA COM^{D} | 29 aa | ERTPSDFSVKGLQMEEMDDIFELLANTLR (SEQ ID NO: 1) |
| | TycB COM^{A} | 14 aa | MSVFSKEQVQDMYA (SEQ ID NO: 2) |
| TycB-C | TycB COM^{D} | 24 aa | EYTPSDLGDEELSMEELENILEWI (SEQ ID NO: 3) |
| | TycC COM^{A} | 12 aa | MKKQENIAKIYP (SEQ ID NO: 4) |
| MlnB-C | MlnB COM^{D} | 31 aa | KNEDQEILFLLEQLKNGELSADQVYQLLEGK |
| | | | (SEQ ID NO: 5) |
| | MInC COM^{A} | 32 33 | MQIKQILSLIEEQQMSPDAGLELIRTYRKEQM |
| | | | (SEQ ID NO: 6) |
| MlnD-E | MlnD COM^{D} | 35 aa | DTADGEIMSLLEKAQTGEMNTKETSELIEELLFH |
| | | | E (SEQ ID NO: 7) |
| | MlnE COM^{A} | 36 aa | MNEKTMQIEQIVKKVKECSLTPEEGLELIKSLGK |
| | | | TH (SEQ ID NO: 8) |
| Kj12B-C | Kj12B COM^{D} | 24 aa | LLKEKRKHFQAEQNSSQEYLRGEI (SEQ ID NO: 9) |
| | Kj12C COM^{A} | 63 aa | MIDAAQIVDEALEQGITLFWNNRLQYETSRDSIP |
| | | | TELLNKWKQHKQELIDFLNQLDSEEQTK (SEQ ID |
| | | | NO: 10) |
| BaeM-N | BaeM COM^{D} | 36 aa | EYTDQEIIAMMKQVSEGTLDFKRVQDIIEGSKTY |
| | | | ES (SEQ ID NO: 11) |
| | BaeN COM^{A} | 32 88 | MNHKELLDAYRSGTLTIAEVEQKLQAFKRTTA |
| | | | (SEQ ID NO: 12) |
| DifF-G | DifF COM^{D} | 34 aa | TSPLETYERLLDELEKNEISIEAAVKRTIGFYRD |
| | | | (SEQ ID NO: 13) |
| | DifG COM^{A} | 33 33 | MKEFLHRVFSDVKNKKMTKQDAIGILRDYDMRM |
| | | | (SEQ ID NO: 14) |
| Ta1-O | Ta1 COM^{D} | 31 aa | EPPSLDELLRQVEAGELDPSVAQQFLTNSQS |
| | | | (SEQ ID NO: 15) |
| | TaO COM^{A} | 34 aa | MTRLEILQALQSGRMALADAKKALAAIEAQVQPE |
| | | | (SEQ ID NO: 16) |
| RizD-E | RizD COM^{D} | 34 aa | PDEDFETLALLRGLSQGALSIEEVDARMTAGMK |
| | | | Q (SEQ ID NO: 17) |
| | RizE COM^{A} | 35 aa | MSGKRTQILQAVQSGAMTPEEGMRQLRALAVA |
| | | | VSE (SEQ ID NO: 18) |

*A donor COM d omain is expressed as COMD and an acceptor COM domain is expressed as COMA. ent of interactions between COM domains
1-2. Measurement of interactions between COM domains

To confirm and quantify interactions between COM domains in the 9 COM domain pairs selected in 1-1, dissociation constants between two domains were measured by fluorescence resonance energy transfer (FRET). FRET refers to an energy transfer that occurs when two fluorescent molecules exist at a distance of a certain distance (less than about 10 nm).

Specifically, a plasmid for expressing a recombinant protein was prepared for the FRET experiment. As the two fluorescent molecules, eGFPd, in which the C-terminus of an enhanced green fluorescent protein (eGFP) was partially deleted, and monomeric red fluorescent protein (mRFP) were used, and it was designed that eGFPd (SEQ ID NO: 24) is fused to the N-terminus of the COMD domain via a (GGGS)2 linker, and mRFP (SEQ ID NO: 25) is fused to the C-terminus of the COMA domain via a (GGGS)2 linker (hereinafter, these fusion proteins are represented as eGFPd-COMD and COMA-mRFP, respectively, with the linkers omitted). The fusion proteins were cloned by using the Gibson assembly method with a vector that expresses the recombinant proteins with a His-tag and protein purification through the affinity chromatography method. To rule out the possibility that the His-tags affect the interaction between the COM domains, the His-tags were linked to the opposite side of the COM domain. eGFPd-COMDs were inserted into pET15b vectors (Novagen), in which a His-tag is added to the N-terminus thereof, and COMA-mRFPs were inserted into pET21c vectors (Novagen) in which a His-tag is added to the C-terminus thereof. Table 2 shows plasmids for expressing recombinant proteins, which were used for in vitro FRET measurement.

TABLE 2

| | | | | | Position of |
|---|---|---|---|---|---|
| COM pair | eGFPd-COM^{D} | COM^{A}-mRFP | Linker | Vector | His-tag |
| TycA-B | eGFPd-TycA | — | (GGGS)₂ | pET15b | N-terminus |
| | COM^{D} | | | | |
| | — | TycB COM^{A}- | (GGGS)₂ | pET21c | C-terminus |
| | | mRFP | | | |
| TycB-C | eGFPd-TycB | — | (GGGS)₂ | pET15b | N-terminus |
| | COM^{D} | | | | |
| | — | TycC COM^{A}- | (GGGS)₂ | pET21c | C-terminus |
| | | mRFP | | | |
| MlnB-C | eGFPd-MlnB | — | (GGGS)₂ | pET15b | N-terminus |
| | COM^{D} | | | | |
| | — | MlnC COM^{A}- | (GGGS)₂ | pET21c | C-terminus |
| | | mRFP | | | |
| MlnD-E | eGFPd-MlnB | — | (GGGS)₂ | pET15b | N-terminus |
| | COM^{D} | | | | |
| | — | MlnC COM^{A}- | (GGGS)₂ | pET21c | C-terminus |
| | | mRFP | | | |
| Kj12B-C | eGFPd-Kj12B | — | (GGGS)₂ | pET15b | N-terminus |
| | COM^{D} | | | | |
| | — | Kj12C COM^{A}- | (GGGS)₂ | pET21c | C-terminus |
| | | mRFP | | | |
| BaeM-N | eGFPd-BaeM | — | (GGGS)₂ | pET15b | N-terminus |
| | COM^{D} | | | | |
| | — | BaeN COM^{A}- | (GGGS)₂ | pET21c | C-terminus |
| | | mRFP | | | |
| DifF-G | eGFPd-DifF COM^{D} | — | (GGGS)₂ | pET15b | N-terminus |
| | — | DifG COM^{A}- | (GGGS)₂ | pET21c | C-terminus |
| | | mRFP | | | |
| Ta-1-O | eGFPd-Ta1 COM^{D} | — | (GGGS)₂ | pET15b | N-terminus |
| | — | TaO COM^{A}- | (GGGS)₂ | pET21c | C-terminus |
| | | mRFP | | | |
| RizD-E | eGFPd-RizD | — | (GGGS)₂ | pET15b | N-terminus |
| | COM^{D} | | | | |
| | — | RizE COM^{A}- | (GGGS)₂ | pET21c | C-terminus |
| | | mRFP | | | |

9 pairs of COM domain fusion fluorescent proteins were obtained by expressing the plasmids shown in Table 2. A certain concentration of eGFPd-COMD was added to 20 mM of Tris-HCl (pH 7.9), 250 mM of NaCl, 10% glycerol, 1 mM of EDTA, and 1 mM of DTT, and samples were prepared with varying concentrations of COMA-mRFP. After the samples were incubated at 37° C. for 30 minutes, fluorescence intensity was measured at one channel (excitation: 488 nm, emission: 605 nm) in a multiwell plate. FRET ratio was calculated by dividing the fluorescent intensity of COMA-mRFP detected in a sample of interest by the fluorescence intensity obtained in a sample containing only eGFPd-COMD. The FRET ratios by COMA-mRFP were plotted and the plots were fitted with the Hill equation to obtain dissociation constants. The interaction between each COM pair was verified and quantified by the dissociation constant, and the results are shown in Table 3.

TABLE 3

| | Dissociation constant |
|---|---|
| COM pair | (K_{d}) |
| TycA-B | 3.99 μM |
| TycB-C | 3.64 μM |
| MlnB-C | 1.08 μM |
| MlnD-E | 1.98 μM |
| Kj12B-C | 6.9 μM |
| BaeM-N | 0.87 μM |
| DifF-G | 2.16 μM |
| Ta-1-O | 2.15 μM |
| RizD-E | 0.67 μM |

1-3. Preparation of In Vitro Metabolon

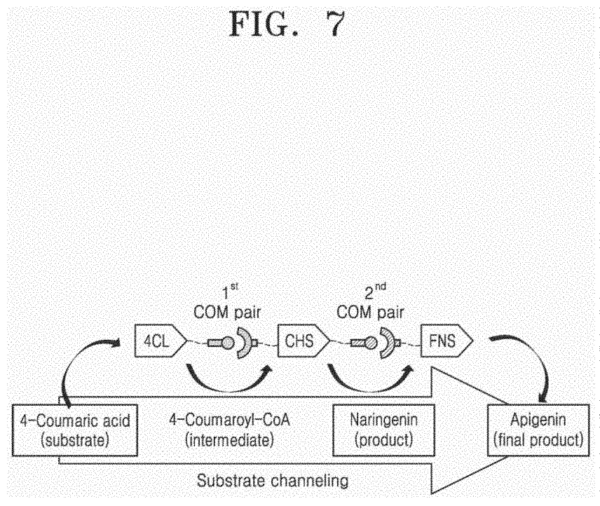

In vitro metabolons for production of a target product were prepared by using some COM domains whose binding affinities were confirmed in 1-2. Specifically, recombinant plasmids were prepared to construct two-step and three-step artificial metabolons with enzymes for biosynthesis of apigenin, a flavonoid-based material. FIG. 7 shows a schematic diagram of three-step 4CL-CHS-FNS metabolon for biosynthesis of apigenin.

In order to produce two-step artificial metabolons, two enzymes, 4-coumarate-CoA ligase (4CL) having an amino acid sequence of SEQ ID NO: 19 and chalcone synthase (CHS) having an amino acid sequence of SEQ ID NO: 20 were used. The COMD domain linked with a (GGGGS)2 linker was designed to be positioned at the C-terminus of 4CL, and the COMA domain linked with a (GGGGS)3 linker was designed to be positioned at the N-terminus of CHS. In addition, for comparative experiments, free 4CL and CHS, that is, 4CL and CHS to which a linker or COM domain is not connected, and 4CL and CHS enzymes to which only a linker is connected were also prepared. For expression of the recombinant proteins and protein purification through the affinity chromatography method, the genes for 4CL-fusion proteins were inserted into pET15b vectors and those for CHS-fusion proteins were inserted into pET21c vectors in the same manner as did for production of eGFPd-COMD and COMA-mRFP. Plasmids for construction of two-step in vitro metabolons are shown in Table 4.

TABLE 4

| | | | | | Position of |
|---|---|---|---|---|---|
| COM pair | 4CL-COM^{D} | COM^{A}-CHS | Linker | Vector | His-tag |
| TycA-B | 4CL-TycA COM^{D} | — | (GGGGS)₂ | pET15b | N-terminus |
| | — | TycB COM^{A}- | (GGGGS)₃ | pET21c | C-terminus |
| | | CHS | | | |
| TycB-C | 4CL-TycB COM^{D} | — | (GGGGS)₂ | pET15b | N-terminus |
| | — | TycC COM^{A}- | (GGGGS)₃ | pET21c | C-terminus |
| | | CHS | | | |
| MlnB-C | 4CL-MlnB COM^{D} | — | (GGGGS)₂ | pET15b | N-terminus |
| | — | MlnC COM^{A}- | (GGGGS)₃ | pET21c | C-terminus |
| | | CHS | | | |
| MlnD-E | 4CL-MlnB COM^{D} | — | (GGGGS)₂ | pET15b | N-terminus |
| | — | MlnC COM^{A}- | (GGGGS)₃ | pET21c | C-terminus |
| | | CHS | | | |
| Kj12B-C | 4CL-Kj12B | — | (GGGGS)₂ | pET15b | N-terminus |
| | COM^{D} | | | | |
| | — | Kj12C COM^{A}- | (GGGGS)₃ | pET21c | C-terminus |
| | | CHS | | | |
| Free | 4CL | — | — | pET15b | N-terminus |
| | — | CHS | — | pET21c | C-terminus |
| Linker | 4CL-linker | — | (GGGGS)₂ | pET15b | N-terminus |
| | — | linker-CHS | (GGGGS)₃ | pET21c | C-terminus |

In addition, in order to produce three-step artificial metabolons, flavone synthase (FNS) having an amino acid sequence of SEQ ID NO: 21 was used along with the two enzymes of 4CL and CHS. In addition to those in the two-step metabolon, recombinant proteins, i.e., CHS having COM domains at both ends, free FNS, FNS having a COMA domain at the N-terminus, and FNS having COM domains at both ends were prepared. In case of MlnC COMA-CHS-TycB COMD and MlnE COMA-FNS-TycA COMD, pGEX-4T-1 vectors (GE Healthcare) were used to express them with a glutathione S-transferase (GST) tag at the N-terminus thereof and the GST tags were removed by treatment with thrombin after obtaining the proteins via affinity chromatography using the GST tag. Genes for the free FNS and COMA-FNS proteins were inserted into pET21c vectors to express them with a His-tag at the C-terminus. In the COMA-FNS protein, COMA and FNS were connected via a (GGGGS)3 linker. Plasmids added for construction of a three-step in vitro metabolon are shown in Table 5.

TABLE 5

| | | | Type and position |
|---|---|---|---|
| Recombinant enzyme | Linker | Vector | of tag |
| MlnC COM^{A}-CHS-TycB | (GGGGS)₃/ | pGEX-4T-1 | GST tag, N-terminus |
| COM^{D} | (GGGGS)₃ | | |
| FNS | — | pET21c | His tag, C-terminus |
| TycC COM^{A}-FNS | (GGGGS)₃ | pET21c | His tag, C-terminus |
| MlnE COM^{A}-FNS | (GGGGS)₃ | pET21c | His tag, C-terminus |
| MlnE COMA-CHS-TycA | (GGGGS)₃/ | pGEX-4T-1 | GST tag, N-terminus |
| COM^{D} | (GGGGS)₃ | | |

The prepared plasmids were introduced into E. coli BL21(DE3) or E. coli Rosetta-gamiTM2(DE3) hosts for protein expression, and treated with Isopropyl β-D-1-thiogalactopyranoside (IPTG) to induce protein expression. Proteins solubilized in the supernatant were separated by centrifugation of the culture medium and purified by affinity chromatography using nitrilotriacetic acid agarose (Ni-NTA agarose) or glutathione sepharose.

### Example 2

### Production of Target Products By Using Artificial Metabolon

2-1. Two-step In Vitro 4CL-CHS Artificial Metabolon

(1) Production of Naringenin

To examine the effect of the artificial metabolon using COM pairs in production of compounds, an in vitro metabolon system was constructed, where it is easy to control concentrations of each enzyme and reaction conditions. Using the recombinant enzymes obtained in Example 1, a metabolon was constructed to perform a 2-step pathway with 4CL and CHS in which naringenin chalcone is synthesized from p-coumaric acid through 4-coumaroyl-CoA, and the naringenin chalcone is spontaneously converted to naringenin.

Specifically, the production of naringenin from p-coumaric acid was compared between 4CL and CHS in free forms and 4CL and CHS connected using any one of 5 pairs of COM domains. In vitro reactions were performed using 4CL and CHS proteins shown in Table 4 in the following combinations. For a single vessel reaction of 4CL and CHS, 1 μM of each of the two enzymes, and 2.5 mM of p-coumaric acid, 200 μM of CoA, and 600 μM of malonyl-CoA, as substrates were added to a buffer of 100 mM of Tris-HCl supplemented with 5 mM of MgCl2, 2.5 mM of ATP, and 1 mM of DTT.

<2-step_control> 4CL and CHS (Free)

<2-step metabolon_1> 4CL-TycA COMD and TycB COMA-CHS (TycA-B)

<2-step metabolon_2> 4CL-TycB COMD and TycC COMA-CHS (TycB-C)

<2-step metabolon_3> 4CL-MlnB COMD and MlnC COMA-CHS (MlnB-C)

<2-step metabolon_4> 4CL-MlnD COMD and MlnE COMA-CHS (MlnD-E)

<2-step metabolon_5> 4CL-Kj12B COMD and Kj12C COMA-CHS (Kj12B-C)

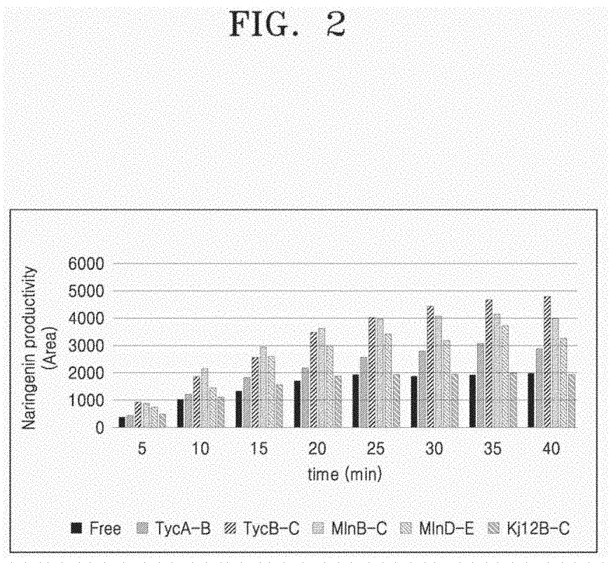

Since each enzyme to which the COM domain is fused is connected to each other at a ratio of 1:1 to form a metabolon, each enzyme was mixed in a 1:1 molar ratio for the reaction. The reactions were carried out at 30° C. for 40 minutes with sampling in a 5 minute interval, and production of naringenin was determined and compared through an UPLC-qTOF-HR-MS analysis. The results are shown in FIG. 2.

Among the 5 pairs of COM domains, productivity by 2-step metabolon_5, Kj12B-C was found similar to that by free enzymes (2-step_control: Free), while the productivity by the metabolons with the remaining four pairs showed improvement attributable to formation of a metabolon, that is, interaction in early reactions (20 minutes or less from the initiation). The production by the reaction using MlnB-C (2-step_metabolon 3), which showed the best effect, was found improved by 2 fold or more compared to that by free enzymes (Free) after 20 minutes of the reaction.

(2) Interaction Between Enzyme Modules

Size exclusion chromatography (SEC) and FRET analysis were performed with the MlnB-C pair to examine the interaction between the two COM domain fusion proteins. The MlnB-C pair showed the highest productivity improvement among the COM domain pairs used for two-step metabolon 4CL-CHS as shown in (1).

Size-exclusion Chromatography Analysis

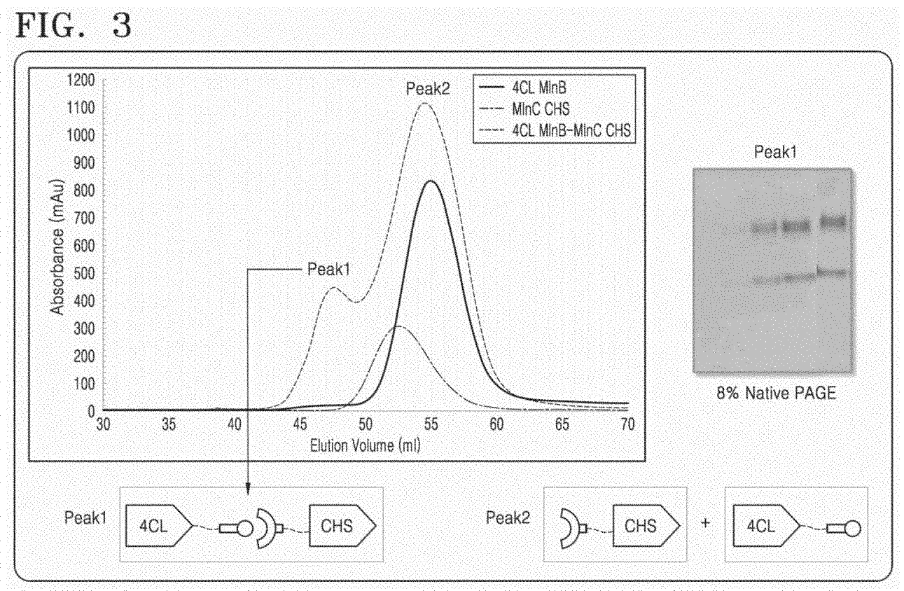

Affinity chromatography, Q ion-exchange chromatography, and size exclusion chromatography (SEC) were sequentially performed on the 4CL-MlnB COMD and MlnC COMA-CHS proteins prepared in (1) to obtain the proteins with high purity. After mixing the two proteins in a 1:1 molar ratio and incubating the mixture at 30° C. for 1 hour, analytical SEC-FPLC (fast protein liquid chromatography) was performed to observe whether they form a complex, in a buffer of 25 mM of Tris-HCl (pH 8.0) supplemented with 150 mM of NaCl, 2 mM of DTT, and 5% glycerol, which is the same as the protein purification buffer. Elution time of each of 4CL-MlnB COMD and MlnC COMA-CHS was measured by analytical SEC. The mixture of the two proteins obtained at the end of the reaction was loaded into a column, and a new peak (designated as Peak1) was observed before the elution volume of each protein. When 4CL and CHS form a complex by COM domains, the resulting protein becomes large. Thus, it was expected that the complex would be eluted faster than each of the proteins, and Peak1 appeared to be the elution peak of the complex. In fact, the two proteins were present in the Peak1 fraction on native PAGE, showing that a complex of 4CL-MlnB COMD and MlnC COMA-CHS was formed. The SEC results are shown in FIG. 3.

FRET Analysis

In addition, interaction between 4CL-MlnB COMD and MlnC COMA-CHS was confirmed by using FRET. It was thought that in 4CL-MlnB COMD and MlnC COMA-CHS, the sizes of the enzymes linked to the COM domains are large, thereby making it difficult to observe FRET occurring at a distance of about 10 nm or less. Therefore, instead of linking fluorescent proteins, Cy3 and Cy5-NHS esters, which are relatively small organic fluorescent molecules, were directly linked to lysine residues naturally occurring in the proteins and FRET was measured at three channels for more accurate measurement.

<Cy3 channel> excitation wavelength: 532 nm, emission wavelength: 570 nm

<Cy5 channel> excitation wavelength: 633 nm, emission wavelength: 670 nm

<FRET channel> excitation wavelength: 532 nm, emission wavelength: 670 nm

Since the emission spectra of Cy3 and Cy5 overlap, some of the fluorescence detected in the FRET channel would be from Cy3, which is referred to as leakage. Similarly, due to the overlap of the excitation spectra of Cy3 and Cy5, Cy5 may be directly excited and thus be detected in the FRET channel, which is called direct excitation. With two fluorescent molecules used, these phenomena inevitably occurred, and additional corrections were made for accurate FRET measurement.

Cy3 leakage was calculated by dividing the fluorescence intensity of the Cy3-labeled 4CL-MlnB COMD in the FRET channel by the fluorescence intensity in the Cy3 channel, and Cy5 direct excitation was calculated by dividing the fluorescence intensity of Cy5-labeled MlnC COMA-CHS in the FRET channel by the fluorescence intensity in the Cy5 channel. After obtaining two correction factors, Cy3 leakage and Cy5 direct excitation, accurate fluorescence intensity in the FRET channel was calculated by subtracting the fluorescence intensity in the Cy3 channel X leakage value and the fluorescence intensity in the Cy5 channel X direct excitation value, from the fluorescence intensity of a given sample in the FRET channel. In order to quantify interaction of the two molecules, FRET efficiency was calculated as corrected fluorescence intensity in the FRET channel/(fluorescence intensity in the Cy3 channel+corrected fluorescence intensity in the FRET channel).

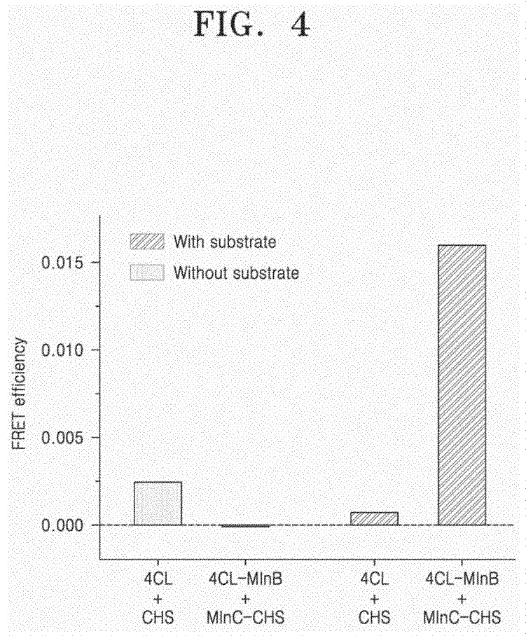

When FRET was measured after 1 μM of each of 4CL-MlnB COMD labeled with Cy3 and Cy5 and MlnC COMA-CHS labeled with Cy3 and Cy5 were added to 100 mM of Tris-HCl (pH 8.0) and reacted at 37° C. for 30 minutes, the FRET efficiency was calculated to be close to zero (without substrate). However, when FRET was measured after 5 mM of MgCl2, 2.5 mM of ATP, and 1 mM of DTT, and as a substrate, 2.5 mM of p-coumaric acid, 200 μM of CoA, and 600 82 M of malonyl-CoA were further added to 100 mM of Tris-HCl (pH 8.0) having the enzymes, and reacted at 37° C. for 30 minutes in the same environment as the enzymatic reaction, the interaction was found with a significant increase in FRET efficiency (with substrate). The results are shown in FIG. 4.

(3) Activity of Enzyme Fused to COM Domain

In an artificial metabolon according to the present disclosure, an enzyme is fused to a COM domain at one end or both ends thereof for selective self-assembly. Thus, it was tested whether the fusion of an enzyme to COM domain(s) affects the activity. A COMA or COMD may be added to the N-terminus/C-terminus of the enzyme, or COMA and COMD may respectively added to the N-terminus and the C-terminus in a process of constructing a metabolon system for a biosynthetic pathway.

For the second enzyme CHS used in the second step of the biosynthesis, the enzyme activity was compared between recombinant proteins COMA-CHS (CHS fused to one COM domain) and COMA-CHS-COMD CHS (CHS fused to two COM domains). To do this, each of the recombinant proteins was mixed with free 4CL enzyme at a ratio of 1:1, and <Reaction 1 and 2 for comparison of CHS activity> were performed as follows by using the single vessel enzyme reaction conditions as described in (1).

<Reaction 1 for comparison of CHS activity> 4CL and MlnC COMA-CHS

<Reaction 2 for comparison of CHS activity> 4CL and MlnC COMA-CHS-TycB COMD

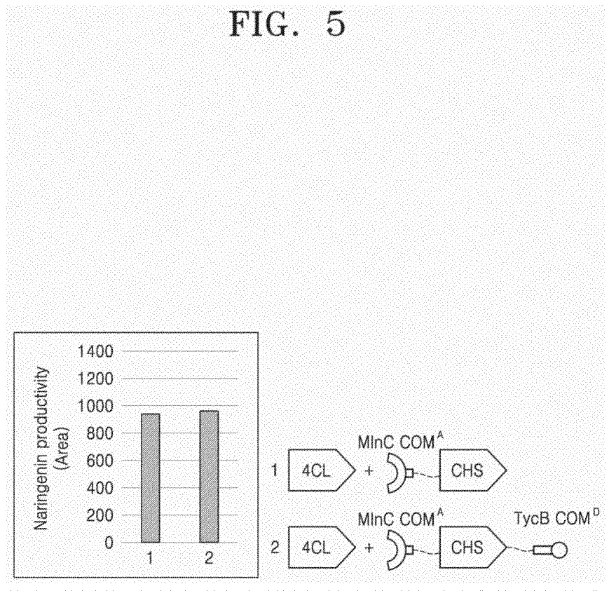

Following the reaction at 30° C. for 15 minutes, production of naringenin measured by an UPLC-qTOF-HR-MS analysis was found almost identical in the two reactions. The results are shown in FIG. 5.

In addition, regarding FNS in the step of converting naringenin, an intermediate produced in a two-step metabolon, into apigenin, a final product, the enzyme activity in a reaction converting the substrate, naringenin to the product, apigenin was compared among free FNS, COMA-FNS, and COMA-CHS-COMD as follows.

<Reaction 1 for comparison of FNS activity> FNS

<Reaction 2 for comparison of FNS activity> TycC COMA-FNS

<Reaction 3 for comparison of FNS activity> MlnE COMA-FNS

<Reaction 4 for comparison of FNS activity> MlnE COMA-FNS-TycA COMD

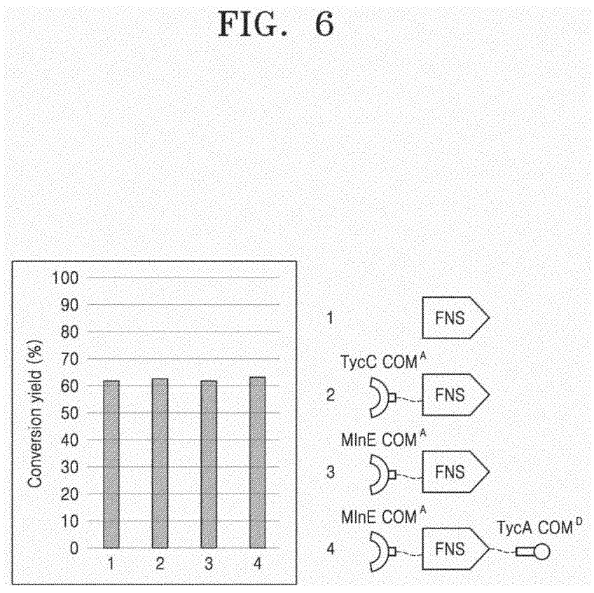

For the reactions, the same amount of an enzyme (50 μg) and 100 μM of naringenin as the substrate were used, and the reactions were performed in 100 mM of Tris-HCl (pH 8.0) buffer supplemented with 5 mM of MgCl2, 10 mM of 2-oxoglutarate, 10 mM of sodium ascorbate, 250 μM of FeSO4, and 1 mM of DTT. After the reactions were carried out at 30° C. for 1 hour, UPLC-qTOF-HR-MS analysis showed about 60% conversion yield in all of free FNS (Reaction 1 for comparison of FNS activity) and three types of COM domain fusion FNSs (Reactions 2, 3, and 4 for comparison of FNS activity). The results are shown in FIG. 6.

Through the two types of reactions for comparing activities, it was confirmed that the type and number of COM domains fused to a biosynthetic enzyme does not significantly affect the activity of the enzyme.

2-2. Three-step 4CL-CHS-FNS Artificial Metabolon

(1) Three-step In Vitro 4CL-CHS-FNS Artificial Metabolon

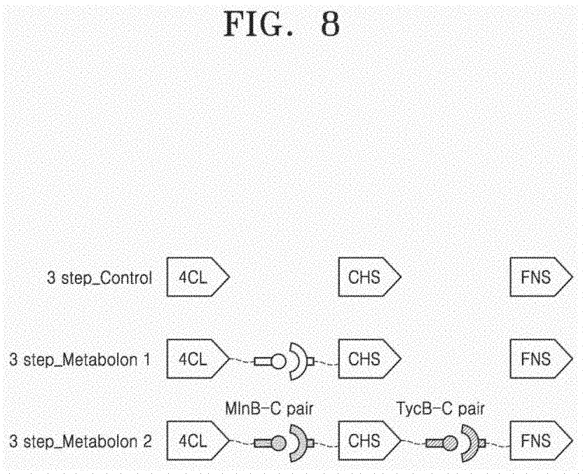

Three-step 4CL-CHS-FNS artificial metabolon was constructed by adding FNS to the two-step 4CL-CHS metabolon constructed in 2-1. FIG. 7 shows a schematic diagram of the three-step 4CL-CHS-FNS metabolon. A biosynthetic pathway was implemented by using a 4-coumarate-CoA ligase (4CL), a chalcone synthase (CHS), and a flavone synthase (FNS), wherein p-coumaric acid, a starting material, is converted through naringenin, an intermediate to apigenin, a final product. Specifically, to confirm the effect of the COM domain pairs identified in (2-1) in actual compound production, 4CL, CHS, and FNS constituting the three-step metabolon were prepared in different combinations as follows: all 4CL, CHS, and FNS as free forms without any fusion to COM domain (3 step_Control), 4CL and CHS as assembled via COM domains and FNS as a free form (3 step_Metabolon 1), and all 4CL, CHS, and FNS as assembled via COM domains (3 step_Metabolon 2). A schematic of the combinations of the recombinant enzymes of 3 step_Control and Metabolons 1 and 2 is shown in FIG. 8.

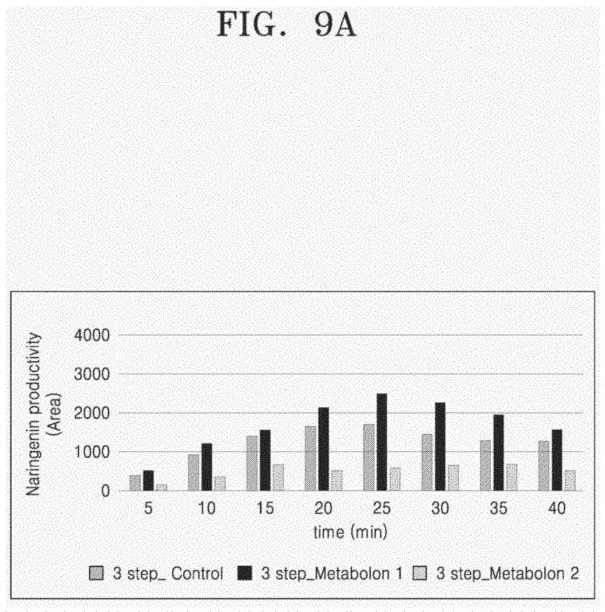

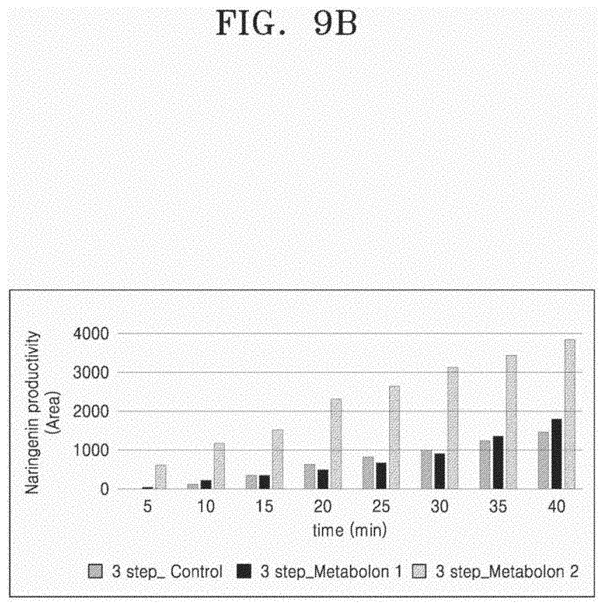

For single vessel enzyme reaction of the three enzymes of 4CL, CHS, FNS, 1 μM of each of the three enzymes, and as a substrate, 2.5 mM of p-coumaric acid, 200 μM of CoA, and 600 μM of malonyl-CoA were used, and the reaction was performed in a buffer of 100 mM of Tris-HCl (pH 8.0) supplemented with 5 mM of MgCl2, 2.5 mM of ATP, 10 mM of 2-oxoglutarate, 10 mM of sodium ascorbate, 250 μM of FeSO4, and 1 mM of DTT. The reaction was carried out at 30° C. for 40 minutes with sampling in a 5 minute interval, and production of naringenin, an intermediate, and apigenin, a final product was determined and compared through an UPLC-qTOF-HR-MS analysis. The results are shown in FIGS. 9A and 9B.

When only the first enzyme and the second enzyme among the three enzymes constituting a three-stage artificial metabolon are assembled by a pair of COM domains (3 step_Metabolon 1), an amount of naringenin, a reaction intermediate, was found to have increased compared to the free enzyme reaction (3 step_Control), however, the efficiency of conversion to apigenin, the final product by FNS was not found to be significantly improved. When all three enzymes were assembled by two pairs of COM domains (3 step_Metabolon 2), the efficiency of conversion of intermediate naringenin to apigenin was increased, the amount of naringenin was found lower than that in 3 step_Control and 3 step_Metabolon 1, and productivity of apigenin, the final product was found to have significantly increased. In the case of the 3 step_Metablon 2, production increased by about 2.6 times after 40 minutes of reaction compared to the free enzymes (3 step_Control), showing that the formation of an artificial metabolon using COM domain pairs is effective in improving production yield in biosynthesis.

(2) Three-step In Vivo 4CL-CHS-FNS Artificial Metabolon

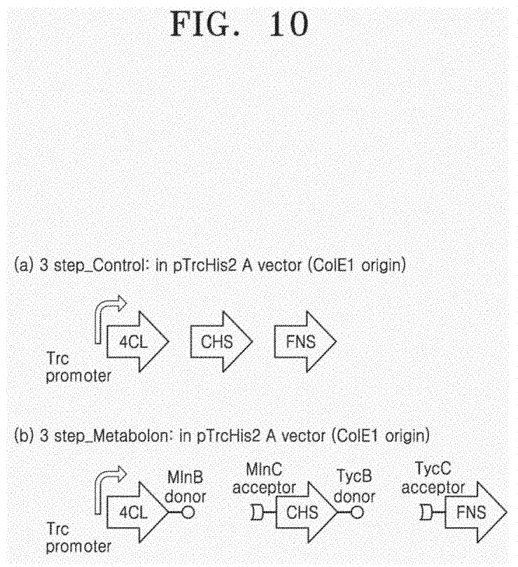

An artificial metabolon 4CL-CHS-FNS for apigenin biosynthesis, which was confirmed to have improved production through in vitro system in (1), was applied in vivo. Using the three-step apigenin biosynthetic pathway (4CL-CHS-FNS), of which metabolon was confirmed to have an effect through an in vitro system, in vivo production of apigenin by the metabolon in E. coli host was made. Three recombinant enzymes 4CL-MlnB COMD, MlnC COMA-CHS-TycB COMD, and TycC COMA-FNS were designed to be expressed in a form of an operon, and inserted into a pTrcHis2 A vector (Invitrogen) having a trc promoter and a ColE1 origin, by the Gibson assembly method (Smith et al. Nat Methods 6(5):343-5 (2009)) to prepare a recombinant vector for expression of three-step_metabolon. Specifically, the recombinant enzyme 4CL consists of 4CL having an amino acid sequence of SEQ ID NO: 19, a (GGGGS)2 linker located at the C-terminus of 4CL, and an MlnB COMD domain linked thereto; the recombinant enzyme CHS consists of CHS having an amino acid sequence of SEQ ID NO: 20, a (GGGGS)3 linker located at the N-terminus of CHS, an MlnC COMA domain linked thereto, a (GGGGS)3 linker located at the C-terminus of CHS, and a TycB COMD domain linked thereto; and the recombinant enzyme FNS consists of FNS having an amino acid sequence of SEQ ID NO: 21, a (GGGGS)3 linker located at the N-terminus of FNS, and a TycC COMA domain linked thereto. For comparative experiments, expression vectors containing free 4CL, CHS, and FNS were prepared in the same manner. FIG. 10 shows schematic diagrams of the expression vectors: (a) 3 step_Control and (b) 3 step_Metabolon.

3 step_Control: 4CL, CHS, and FNS in a pTrcHis2 A vector

3 step_Metabolon: 4CL-MlnB COMD, MlnC COMA-CHS-TycB COMD, and TycC COMA-FNS in a pTrcHis2 A vector

The expression vectors were introduced into E. coli K-12 MG1655, and production of apigenin was determined in liquid culture. For pre-culture, the transformants were inoculated into 3 mL of Luria-Bertani (LB) liquid medium supplemented with 50 μg/mL of ampicillin, incubated overnight at 37° C. followed by reinoculation into 50 mL of LB liquid medium (50 μg/mL ampicillin) to OD600 of ˜0.1, and additional incubation at 37° C. until OD600 reached ˜0.7. For main culture, the transformants obtained from the pre-culture were reinoculated into 50 mL of YM9 (6.8 g Na2HPO4, 3 g KH2PO4, 0.5 g NaCl, 1 g NH4Cl, 10 g yeast extract, 30 mL glycerol, 42 g 3-(N-morpholino)propanesulfonic acid (MOPS), pH 7.0, per 1L) liquid medium (50 μg/mL of ampicillin) to OD600 of ˜0.1 and incubated at 37° C. until OD600 reached ˜0.7. Thereafter, 1 mM of IPTG for induction of expression and 1 mM of p-coumaric acid, which is used as a substrate, were added, and incubated at 30° C. for about 40 hours. As a result, when the free enzymes were expressed (3 step_Control), about 40 μg/L of apigenin was produced, and when the enzymes assembled by the COM domains (3 step_Metabolon) were expressed, about 610 μg/L of apigenin was produced, which is about 15 times that of 3 step_Control.

2-3. Two-step In Vivo CNL-AAL Artificial Metabolon

Using the COM domains whose binding affinities were confirmed in Example 1, an in vivo metabolon for shinorine biosynthesis, a type of mycosporine-like amino acid (MAA), was prepared. Specifically, a plasmid was prepared to construct an in vivo metabolon for biosynthesis of shinorine, an MAA, in Saccharomyces cerevisiae.

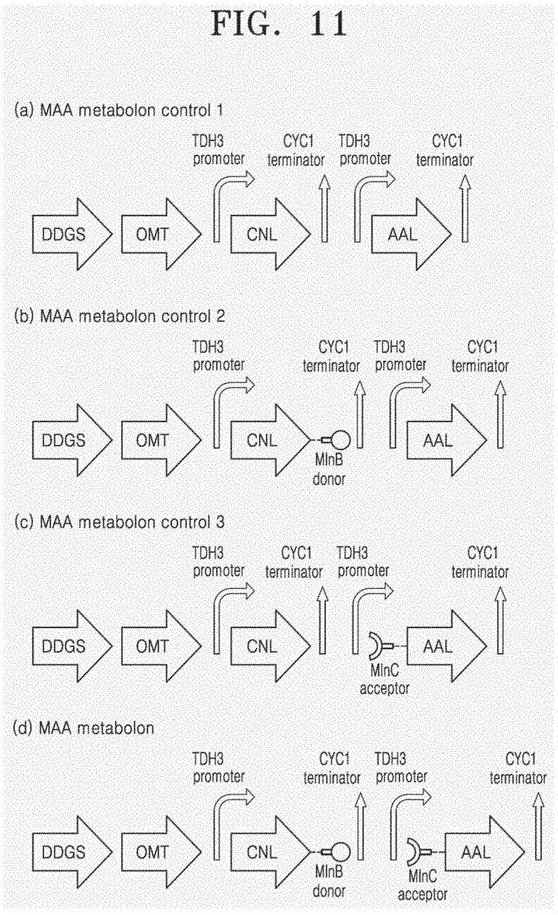

For the preparation of a metabolon for shinorine biosynthesis, among the four enzymes required for shinorine biosynthesis (2-desmethyl 4-deoxygadusol synthase (DDGS), O-methyltransferase (OMT), ATP-grasp ligase (CNL), and D-Alanine D-Alanine ligase (AAL)), two enzymes, CNL having an amino acid sequence of SEQ ID NO: 22, and AAL having an amino acid sequence of SEQ ID NO: 23, were assembled by COM domains. It was designed that a MlnB COMD domain linked to a (GGGS) linker was positioned at the N-terminus of CNL and a MlnC COMA domain linked to a (GGGGS)2 linker was positioned at the N-terminus of AAL. In addition, for comparison, free CNL and AAL, that is, CNL and AAL to which a linker or a COM domain is not connected, were prepared. Two additional enzymes, DDGS (SEQ ID NO: 26) and OMT (SEQ ID NO: 27), required for shinorine biosynthesis were constructed to be expressed under the same promoter and terminator but without any linker or COM domain. The proteins were expressed by using a CEN/ARS vector with a TDH3 promoter, a CYC1 terminator and a URA3 marker. FIG. 11 shows schematic diagrams of the expression vectors.

<MAA metabolon control 1> DDGS, OMT, CNL, AAL in CEN/ARS vector

<MAA metabolon control 2> DDGS, OMT, MlnB COMD-CNL, AAL in CEN/ARS vector

<MAA metabolon control 3> DDGS, OMT, CNL, MlnC COMA-AAL in CEN/ARS vector

<MAA metabolon> DDGS, OMT, MlnB COMD-CNL, MlnC COMA-AAL in CEN/ARS vector

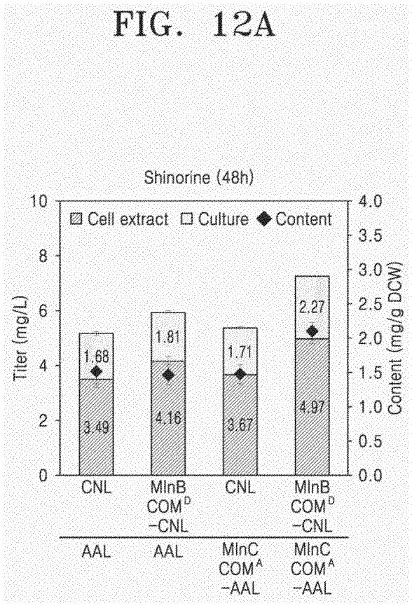

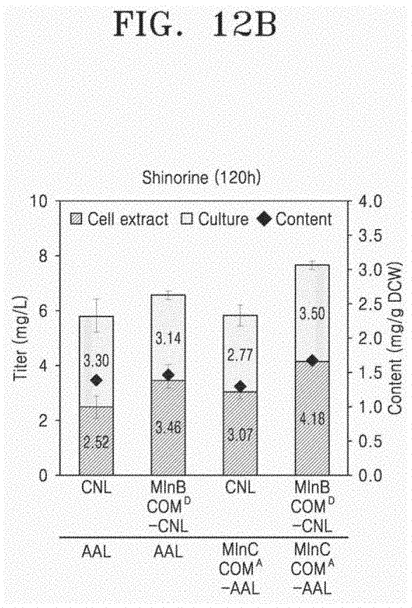

Shinorine production was conducted by introducing the prepared expression vector into a Saccharomyces cerevisiae strain (Park S H et al. ACS Synth Biol. 8(2):346-57 (2019)) with xylose consuming ability and cultivating the strains in liquid media. For primary culture, the strain was inoculated into 5 mL of uracil-deficient synthetic complete (SC) medium (6.7 g/L of yeast nitrogen base (YNB) without amino acids, and 1.92 g/L of amino acids dropout mixture lacking uracil) and cultured for 24 hours at 30° C. and 170 rpm. The primary culture obtained was inoculated into 10 mL of the same medium to an initial OD600 of ˜0.2, and incubated at 30° C. and 170 rpm for the secondary culture. 10 g/L of glucose and 10 g/L of xylose were used as carbon sources of the medium. After 48 hours and 120 hours of the secondary culture, the culture and the cell extract were analyzed by high performance liquid chromatography (HPLC) to measure shinorine production. As a result, when CNL and AAL linked by COM domains were expressed (MAA metabolon), shinorine production increased by about 1.3 times compared to when CNL and AAL as free forms were expressed (MAA metabolon control 1), and when only one of CNL and AAL fused to COMD domain or COMA domain and the other as free from were expressed (MAA metabolon control 2 and 3). The results are shown in FIG. 12.

What is claimed is:

1. An artificial metabolon formed by selective self-assembly, comprising 2 or more enzyme modules,
which is represented by (Xi)n, wherein
Xi is a module consisting of N-terminal communication-mediating (COM) domain-recombinant enzyme-C-terminal COM domain,
i is an integer equal to or higher than 1, n is an integer equal to or higher than 2,
one of the N-terminal COM domain and the C-terminal COM domain is a donor COM domain (COM^{D}) and the other is an accepter COM domain (COM^{A}),
the donor COM domain (COM^{D}) of the (i)th enzyme module Xi specifically binds to an acceptor COM domain (COM^{A}) of the (i+1)th enzyme module Xi+1 so that all the enzymes from X1, a first enzyme module, to Xn, the nth enzyme module, are connected to form an artificial metabolon by selective self-assembly,
the first enzyme module X1 (i=1) of the artificial metabolon does not include any acceptor COM domain (COM^{A}), and the nth enzyme module (i=n) does not include any donor COM domain (COM^{D}), and
the reaction product produced by the recombinant enzyme of the (i)th enzyme module Xi is used as a substrate for the recombinant enzyme of the (i+1)th enzyme module Xi+1.

2. The artificial metabolon of claim 1, wherein each enzyme module of the artificial metabolon comprises a linker connecting the recombinant enzyme and the COM domains.

3. The artificial metabolon of claim 1, wherein the donor COM domain (COM^{D}) of the enzyme module Xi and the acceptor COM domain (COM^{A}) of the enzyme module Xi+1 have a predetermined binding affinity (Kd).

4. The artificial metabolon of claim 1, wherein a pair of a donor COM domain (COM^{D}) and an acceptor COM domain (COM^{A}) which specifically bind to each other are derived from the COM domains of nonribosomal peptide synthetase (NRPS) or the docking domains of trans-AT polyketide synthase (PKS).

5. The artificial metabolon of claim 4, wherein the pair of donor COM domain (COM^{D}) and acceptor COM domain (COM^{A}) is selected from the group consisting of TycA-TycB, TycB-TycC, MlnB-MlnC, MlnD-MlnE, Kj12B-Kj12C, BaeM-BaeN, DifF-DifG, Ta-1-TaO, and RizD-RizE.

6. The artificial metabolon of claim 1, wherein the artificial metabolon comprises enzyme modules for the enzymes constituting a metabolic pathway for biosynthesis of a target product and is capable of producing the target product from a starting substrate for the target product.

7. The artificial metabolon of claim 1, wherein the artificial metabolon comprises multiple identical enzyme modules.

8. An artificial metabolon for biosynthesis of apigenin, comprising:
a recombinant enzyme 4-coumarate CoA ligase (4CL) having a first COM domain at the N-terminus or C-terminus, a recombinant enzyme chalcone synthase (CHS) having a second COM domain and a third COM domain at the N-terminus and C-terminus, respectively, and a recombinant enzyme flavone synthase (FNS) having a fourth COM domain at the N-terminus,
wherein the first COM domain specifically binds to the second COM domain, and the third COM domain specifically binds to the fourth COM domain.

9. The artificial metabolon of claim 8, wherein the artificial metabolon further comprises a linker between the COM domain and the recombinant enzyme.

10. The artificial metabolon of claim 8, wherein the pair of the first COM domain and the second COM domain and the pair of the third COM domain and the fourth COM domain are a MlnB-C pair and a TycB-C pair, respectively.

11. A recombinant vector comprising a nucleic acid encoding the artificial metabolon of claim 1, wherein the recombinant vector comprises an operon in which genes respectively encoding each enzyme module are operably linked to expression regulatory sequences.

12. The recombinant vector of claim 11, wherein the recombinant vector is configured to regulate expression levels in consideration of stoichiometry of the enzymes constituting the artificial metabolon.

13. A cell transformed with the recombinant vector comprising a nucleic acid encoding the artificial metabolon of claim 1.

14. A method of producing a target product by using the artificial metabolon of claim 1, comprising:
contacting a substrate for the recombinant enzyme in the first enzyme module of the artificial metabolon with the artificial metabolon.

15. The method of claim 14, wherein the method is performed in vitro.

16. The method of claim 14, wherein the method comprises culturing cells transformed with the nucleic acid encoding the artificial metabolon in the presence of the substrate.

17. The method of claim 14, wherein the target product is amino acids, peptides, proteins, lipids, monosaccharides, polysaccharides, nucleic acids, or compounds.


SEQUENCE LISTING (27 sequences)

SEQ ID NO: 1 - protein, 29 aa, Bacillus brevis
ERTPSDFSVKGLQMEEMDDIFELLANTLR

SEQ ID NO: 2 - protein, 14 aa, Bacillus brevis
MSVFSKEQVQDMYA

SEQ ID NO: 3 - protein, 24 aa, Bacillus brevis
EYTPSDLGDEELSMEELENILEWI

SEQ ID NO: 4 - protein, 12 aa, Bacillus brevis
MKKQENIAKIYP

SEQ ID NO: 5 - protein, 31 aa, Bacillus amyloliquefaciens
KNEDQEILFLLEQLKNGELSADQVYQLLEGK

SEQ ID NO: 6 - protein, 32 aa, Bacillus amyloliquefaciens
MQIKQILSLIEEQQMSPDAGLELIRTYRKEQM

SEQ ID NO: 7 - protein, 35 aa, Bacillus amyloliquefaciens
DTADGEIMSLLEKAQTGEMNTKETSELIEELLFHE

SEQ ID NO: 8 - protein, 36 aa, Bacillus amyloliquefaciens
MNEKTMQIEQIVKKVKECSLTPEEGLELIKSLGKTH

SEQ ID NO: 9 - protein, 24 aa, Xenorhabdus stockiae
LLKEKRKHFQAEQNSSQEYLRGEI

SEQ ID NO: 10 - protein, 63 aa, Xenorhabdus stockiae
MIDAAQIVDEALEQGITLFVVNNRLQYETSRDSIPTELLNKWKQHKQELIDFLNQLDSEEQTK

SEQ ID NO: 11 - protein, 36 aa, Bacillus amyloliquefaciens
EYTDQEIIAMMKQVSEGTLDFKRVQDIIEGSKTYES

SEQ ID NO: 12 - protein, 32 aa, Bacillus amyloliquefaciens
MNHKELLDAYRSGTLTIAEVEQKLQAFKRTTA

SEQ ID NO: 13 - protein, 34 aa, Bacillus amyloliquefaciens
TSPLETYERLLDELEKNEISIEAAVKRTIGFYRD

SEQ ID NO: 14 - protein, 33 aa, Bacillus amyloliquefaciens
MKEFLHRVFSDVKNKKMTKQDAIGILRDYDMRM

SEQ ID NO: 15 - protein, 31 aa, Myxococcus xanthus
EPPSLDELLRQVEAGELDPSVAQQFLTNSQS

SEQ ID NO: 16 - protein, 34 aa, Myxococcus xanthus
MTRLEILQALQSGRMALADAKKALAAIEAQVQPE

SEQ ID NO: 17 - protein, 34 aa, Stigmatella aurantiaca
PDEDFETLALLRGLSQGALSIEEVDARMTAGMKQ

SEQ ID NO: 18 - protein, 35 aa, Stigmatella aurantiaca
MSGKRTQILQAVQSGAMTPEEGMRQLRALAVAVSE

SEQ ID NO: 19 - protein, 561 aa, Arabidopsis thaliana
MAPQEQAVSQVMEKQSNNNNSDVIFRSKLPDIYIPNHLSLHDYIFQNISEFATKPCLINGPTGHVYTYSDVHVISRQIAANFHKLGVNQNDVVMLLLPNCPEFVLSFLAASFRGATATAANPFFTPAEIAKQAKASNTKLIITEARYVDKIKPLQNDDGVVIVCIDDNESVPIPEGCLRFTELTQSTTEASEVIDSVEISPDDVVALPYSSGTTGLPKGVMLTHKGLVTSVAQQVDGENPNLYFHSDDVILCVLPMFHIYALNSIMLCGLRVGAAILIMPKFEINLLLELIQRCKVTVAPMVPPIVLAIAKSSETEKYDLSSIRVVKSGAAPLGKELEDAVNAKFPNAKLGQGYGMTEAGPVLAMSLGFAKEPFPVKSGACGTVVRNAEMKIVDPDTGDSLSRNQPGEICIRGHQIMKGYLNNPAATAETIDKDGWLHTGDIGLIDDDDELFIVDRLKELIKYKGFQVAPAELEALLIGHPDITDVAVVAMKEEAAGEVPVAFVVKSKDSELSEDDVKQFVSKQVVFYKRINKVFFTESIPKAPSGKILRKDLRAKLANGL

SEQ ID NO: 20 - protein, 389 aa, Petunia x hybrida
MVTVEEYRKAQRAEGPATVMAIGTATPSNCVDQSTYPDFYFRITNSEHKTDLKEKFKRMCEKSMIKKRYMHLTEEILKENPSMCEYMAPSLDARQDIVVVEVPKLGKEAAQKAIKEWGQPKSKITHLVFCTTSGVDMPGCDYQLTKLLGLRPSVKRLMMYQQGCFAGGTVLRLAKDLAENNKGARVLVVCSEITAVTFRGPNDTHLDSLVGQALFGDGAGAIIIGSDPIPGVERPLFELVSAAQTLLPDSHGAIDGHLREVGLTFHLLKDVPGLISKNIEKSLEEAFKPLGISDWNSLFWIAHPGGPAILDQVEIKLGLKPEKLKATRNVLSNYGNMSSACVLFILDEMRKASAKEGLGTTGEGLEWGVLFGFGPGLTVETVVLHSVAT

SEQ ID NO: 21 - protein, 365 aa, Petroselinum crispum
MAPTTITALAKEKTLNLDFVRDEDERPKVAYNQFSNEIPIISLAGLDDDSDGRRPEICRKIVKACEDWGIFQVVDHGIDSGLISEMTRLSREFFALPAEEKLEYDTTGGKRGGFTISTVLQGDDAMDWREFVTYFSYPINARDYSRWPKKPEGWRSTTEVYSEKLMVLGAKLLEVLSEAMGLEKGDLTKACVDMEQKVLINYYPTCPQPDLTLGVRRHTDPGTITILLQDMVGGLQATRDGGKTWITVQPVEGAFVVNLGDHGHYLSNGRFRNADHQAVVNSTSSRLSIATFQNPAQNAIVYPLKIREGEKAILDEAITYAEMYKKCMTKHIEVATRKKLAKEKRLQDEKAKLEMKSKSADENLA

SEQ ID NO: 22 - protein, 461 aa, Nostoc punctiforme
MAQSISLSLPQSTTPSKGVRLKIAALLKTIGTLILLLIALPLNALIVLISLMCRPFTKKPAVATHPQNILVSGGKMTKALQLARSFHAAGHRVILIEGHKYWLSGHRFSNSVSRFYTVPAPQDDPEGYTQALLEIVKREKIDVYVPVCSPVASYYDSLAKSALSEYCEVFHFDADITKMLDDKFAFTDRARSLGLSAPKSFKITDPEQVINFDFSKETRKYILKSISYDSVRRLNLTKLPCDTPEETAAFVKSLPISPEKPWIMQEFIPGKELCTHSTVRDGELRLHCCSNSSAFQINYENVENPQIQEWVQHFVKSLRLTGQISLDFIQAEDGTAYAIECNPRTHSAITMFYNHPGVAEAYLGKTPLAAPLEPLADSKPTYWIYHEIWRLTGIRSGQQLQTWFGRLVRGTDAIYRLDDPIPFLTLHHWQITLLLLQNLQRLKGWVKIDFNIGKLVELGGD

SEQ ID NO: 23 - protein, 367 aa, Lyngbya sp. PCC 8106
MKLETIRPTQPLSSDTLPILRVLHLAGSCVSDFYYNLSIVYAKEVVQPVGVSSYYAVVHPNGDWQLGTSLDSLSEKMSLQEMIVQLPEIDVVVPHQFCFPGMTSFRAFFEDLLGLSVVGSPSHCTALATNKAQTRSVVAASGVRVAKAQQLRPGDTLTMQPPFIVKPNSEDNSLGVTLVQNPDQITEALRIGFELDEILLVEDYIPGREIRVAVVEREGKLCVLPMIEYLFTEEHPIRTVQDKLHLQSDGMPEKQPEKPVAKPVCPANVAPELFEKLANAAKNAHIALGCRDYSLYDFRVHAETHEPYMLEAGLFWSFGKISMISRMLIVDGQNLEDVALELWREAAGRTRVACRSLFKYTKANQLV

SEQ ID NO: 24 - protein, 228 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAA

SEQ ID NO: 25 - protein, 224 aa, synthetic construct
ASSEDVIKEFMRFKVRMEGSVNGHEFEIEGEGEGRPYEGTQTAKLKVTKGGPLPFAWDILSPQFQYGSKAYVKHPADIPDYLKLSFPEGFKWERVMNFEDGGVVTVTQDSSLQDGEFIYKVKLRGTNFPSDGPVMQKKTMGWEASTERMYPEDGALKGEIKMRLKLKDGGHYDAEVKTTYMAKKPVQLPGAYKTDIKLDITSHNEDYTIVEQYERAEGRHSTGA

SEQ ID NO: 26 - protein, 410 aa, Nostoc punctiforme
MSNVQASFEATEAEFRVEGYEKIEFSLVYVNGAFDISNREIADSYEKFGRCLTVIDANVNRLYGKQIKSYFRHYGIDLTVVPIVITEPTKTLATFEKIVDAFSDFGLIRKEPVLVVGGGLTTDVAGFACAAYRRKSNYIRVPTTLIGLIDAGVAIKVAVNHRKLKNRLGAYHAPLKVILDFSFLQTLPTAQVRNGMAELVKIAVVANSEVFELLYEYGEELLSTHFGYVNGTKELKAIAHKLNYEAIKTMLELETPNLHELDLDRVIAYGHTWSPTLELAPMIPLFHGHAVNIDMALSATIAARRGYITSGERDRILSLMSRIGLSIDHPLLDGDLLWYATQSISLTRDGKQRAAMPKPIGECFFVNDFTREELDAALAEHKRLCATYPRGGDGIDAYIETQEESKLLGV

SEQ ID NO: 27 - protein, 277 aa, Nostoc punctiforme
MTSILGRDTARPITPHSILVAQLQKTLRMAEESNIPSEILTSLRQGLQLAAGLDPYLDDCTTPESTALTALAQKTSIEDWSKRFSDGETVRQLEQEMLSGHLEGQTLKMFVHITKAKSILEVGMFTGYSALAMAEALPDDGRLIACEVDSYVAEFAQTCFQESPHGRKIVVEVAPALETLHKLVAKKESFDLIFIDADKKEYIEYFQIILDSHLLAPDGLICVDNTLLQGQVYLPSEQRTANGEAIAQFNRIVAADPRVEQVLLPIRDGITLIRRLV